(19) United States
(12) Patent Application Publication
(10) Pub. No.: US 2022/0152203 A1
(43) Pub. Date: May 19, 2022

(54) BROAD-BASED THERAPEUTIC FOR INACTIVATING COVID-19 AND OTHER VIRAL PATHOGENS USING CHEMILUMINESCENCE-ACTIVATED COMPOUNDS ENCAPSULATED IN NANOPARTICLES

(71) Applicant: Trevor Percival Castor, Arlington, MA (US)

(72) Inventor: Trevor Percival Castor, Arlington, MA (US)

(73) Assignee: Trevor P. Castor, Woburn, MA (US)

(21) Appl. No.: 17/526,693

(22) Filed: Nov. 15, 2021

Related U.S. Application Data
(60) Provisional application No. 63/113,937, filed on Nov. 15, 2020.

(51) Int. Cl. A61K 41/00; A61K 9/127; A61K 31/122; A61K 38/46; A61K 31/785; A61K 9/107; A61K 9/19; A61K 31/352; A61P 31/14; A61P 31/18

(52) U.S. Cl. CPC A61K 41/0057; A61K 9/1271; A61K 31/122; A61K 38/465; C12Y 301/03001; A61P 31/18; A61K 9/1075; A61K 9/19; A61K 31/352; A61P 31/14; A61K 31/785

(57) ABSTRACT

Method and apparatus for producing a broad-spectrum therapeutic for inactivating multiple coronavirus strains including COVID-19. The present invention generates an in situ light source which can inactivate the coronavirus within the body without damaging host cells. This invention couples chemiluminescence-generating compounds with various light-sensitive anti-viral compounds. When activated by chemiluminescence-generated light, the anti-viral compounds inactivate nearby virus particles. In one aspect, the coupled components are co-encapsulated in polymer nanospheres for oral and intranasal delivery. In another aspect of the invention, the coupled components are co-encapsulated in phospholipid nanosomes for intravenous delivery.

### FIELD OF INVENTION

The present invention relates to chemiluminescence-directed antiviral activities of natural and synthesized light-sensitive compounds. Specifically, the invention herein describes methods for inactivating infectious virus particles outside and inside an organism, in the latter case being used as a treatment for viral diseases such as COVID-19.

### REFERENCES TO OTHER PATENTS

This application discloses a number of improvements and enhancements to the anti-viral complexes and methods disclosed in U.S. Pat. No. 7,027,524 to Castor et al., which is hereby incorporated by reference in its entirety.

This application discloses a number of improvements and enhancements to the apparatus and methods disclosed in U.S. Pat. No. 9,981,238 to Castor, which is hereby incorporated by reference in its entirety.

This application discloses a number of improvements and enhancements to the apparatus and methods disclosed in U.S. Pat. No. 5,776,486 to Castor et al., which is hereby incorporated by reference in its entirety.

### BACKGROUND OF THE INVENTION

The United States and the World are currently experiencing an infectious disease pandemic caused by a novel coronavirus SARS-CoV-2 that causes COVID-19. As of this writing (Nov. 12, 2021), there are more than 252,340,354 confirmed cases worldwide and 5,085,702 fatalities (a confirmed case fatality rate of 2.0%). In the United States, at this time (Nov. 12, 2021), there are more than 46,931,055 confirmed cases and 760,970 fatalities (a confirmed case fatality rate of 1.6%). Apart from its significant morbidity and mortality, this pandemic has had a significant impact on the fabric of the society and the US economy, causing over 20 million in unemployment, trillions in loss revenue, and the most dramatic downturn in the Gross Domestic Product since the Great Depression of the 1920's. These statistics are unfortunately very fluid since the pandemic is ongoing even with mass scale vaccinations and monoclonal antibody therapeutics in addition to containment, mitigation, and supportive respiratory care.

The novel coronavirus, COVID-19, emerged in Wuhan, China in late December 2019. Coronaviruses are a large family of viruses that may cause illness in animals and humans. In humans, several coronaviruses are known to cause respiratory disease such as Middle East Respiratory Syndrome (MERS), Severe Acute Respiratory Syndrome (SARS) and the most recently discovered COVID-19. These viruses are all genetically related with both SARS (10% fatality rate) and MERS (37.4% fatality rate) being more deadly than COVID-19 but much less infectious.

Several compounds with broad anti-viral activity have been described in the literature, but most have been unsuitable for commercial development because of toxicity to the patient. Industry and governments have responded to the current pandemic with an urgent and successful development of vaccines and emerging therapeutics against COVID-19.

There remains an urgent need for a broad-spectrum therapeutic for multiple coronavirus strains including the virus and its variants that are the etiologic agents of COVID-19.

### SUMMARY OF THE INVENTION

The present invention is a broad-spectrum therapeutic for multiple coronavirus strains including SARS-CoV-2 and its variants that cause COVID-19. The present invention generates an in situ light source which can inactivate the coronavirus within the body without damaging host cells. This invention couples chemiluminescence-generating compounds with various light-sensitive anti-viral compounds. When activated by chemiluminescence, the anti-viral compounds inactivate virus particles. Infectious virus particles may be inactivated outside an organism, or in situ, inside an organism, providing a treatment for viral diseases.

The present inventor and other researchers have demonstrated that a natural plant product—hypercin—when activated by a light source, provides a high level of viral inactivation. Relatively short exposure times, which occur during routine tissue culture infection procedures, were sufficient for near complete inactivation of the exposed virus, notably HIV and other retroviruses.

However, the virucidal effects of hypericin are minimal if the virus is treated in complete darkness, as in the body. In order to generate an in situ light source, the present invention uses what is known as a “molecular flashlight” [U.S. Pat. No. 7,027,525 to Castor et al]. Chemiluminescent substrates combined with enzymes, such as alkaline phosphatase, and emission enhancers or anti-quenchers emit sufficient light to “turn on” the anti-viral properties of the hypericin. It has been demonstrated that hypericin in the presence of this “molecular flashlight” can significantly inactivate HIV in the dark (10⁶ TCID₅₀) in cell culture media in vitro without harming these cells.

In one aspect of the present invention, multiple coronavirus strains are inactivated by a combination of hypericin, chemiluminescent substrates, alkaline phosphatase enzymes and emission enhancers (or anti-quenchers). The components are optimized during in vitro efficacy and cytotoxicity studies against coronaviruses including COVID-19, and formulations are developed for the delivery of the broad-spectrum antiviral coronavirus therapeutic. The formulations include dry powder pills, gel capsules, and nanoparticles—both phospholipid nanosomes for intravenous delivery, and polymer nanospheres for oral delivery. The formulations are evaluated for in vitro performance (release characteristics, stability, efficacy and toxicity) to inform in vivo studies and optimize clinical development. The results of these studies provide for accelerated development of the present broad-spectrum therapeutic for multiple coronavirus strains and SARS-CoV-2.

In another aspect of the present invention, in vivo experiments in animal models of coronavirus are conducted to establish pharmacokinetics, toxicity, and efficacy, to further demonstrate that manufacturing can be scaled in accordance with cGMP guidelines. This provides a regulatory pathway with the FDA to conduct clinical trials on the broad-based, anti-coronavirus therapeutic leading to commercialization and stockpiling.

A further aspect of the present invention is a broad-based therapeutic for COVID-19 and other viral pathogens, which is scalable for large quantity manufacturing and distribution.

A yet another aspect, the therapeutic for COVID-19 and other viral pathogens can be delivered to patients intravenously using phospholipid nanosomes.

A yet another aspect, the therapeutic for COVID-19 and other viral pathogens can be delivered to patients orally using polymer nanospheres.

A yet another aspect, the therapeutic for COVID-19 and other viral pathogens can be delivered to patients intranasally using phospholipid nanosomes and/or polymer nanospheres.

These and other features, aspects and advantages of the present teachings are better understood with reference to the following description, examples, and appended claims.

### BRIEF DESCRIPTION OF THE DRAWINGS

FIG. 1 shows a scanning electron microscopic image of coronavirus, SARS-CoV-2;

FIG. 2 is a schematic representation of chemiluminescence-directed inactivation of the SARS-CoV-2 virus that causes COVID-19;

FIG. 3 is a graph depicting inhibition of HIV growth in infected cultures by concerted action of hypericin, chemiluminescent substrate and alkaline phosphatase;

FIG. 4 illustrates the chemical structure of hypericin;

FIG. 5 illustrates the chemical structure of CDP Star, a chemiluminescent substrate manufactured by Topex Inc, and used in the present invention.

FIG. 6 illustrates the chemical structure of D-Luciferin, another chemiluminescent substrate used in the present invention;

FIG. 7 illustrates the process of chemiluminescent enhancement according to the present invention; and

FIG. 8 schematically shows the SuperFluids™ apparatus of the present invention used to create phospholipid nanosomes for encapsulating the broad spectrum therapeutic of the present invention according to the present invention.

FIG. 9 is a graph illustrating the inactivation of HIV-1 replication by the concerted action of hypericin and visible light. Infection of CEM-TART cells with HIV-Δtat/Δrev strain was performed for 1 hour with 50 TCID₅₀ upon exposure of viral stock to 5 μM of hypericin and visible light (solid squares) or dark (open squares) for 1 hours at 37° C. Untreated virus was also used to infect CEM-TART cells at 50 TCID₅₀ (solid circles) after a 2-hour incubation at 37° C. Greater than 99% of the virus was inactivated when exposed to hypericin and light, even after 20 days of culture;

FIG. 10 is a graph showing the virucidal effect of hypericin and some chemiluminescence-inducing compounds such as alkaline phosphatase and CSPD/Emerald. Approximately 50 TCID₅₀ of HIVΔtat/Δrev viral stocks were pre-treated with a mixture of hypericin, chemiluminescence substrate with enhancer and/or alkaline phosphatase (AP) for 1 hour at 37° C. and then used to infect CEM-TART cells. HIV p24 was monitored over time. Data is the average of duplicate samples. Open circles, virus+hypericin+enhancer+AP+dark; solid circles, virus+hypericin+enhancer+dark; open squares, virus+hypericin+AP+dark; solid squares, untreated virus; solid triangles, virus+hypericin+visible light. Data is the average of duplicate samples. Incubating virus with hypericin, alkaline phosphatase and a chemiluminescent substrate with enhancer results in equivalent virus inactivation as when hypericin and visible light are used (99.3% virus reduction with chemiluminescent substrate and enhancer versus 99.4% virus reduction with visible light on day 7 post-infection);

FIG. 11 is a graph depicting the level of virucidal effect of chemiluminescence-induced hypericin action as a function of the concentration of the active enzyme. Approximately 50 TCID₅₀ of HIVΔtat/Δrev viral stocks were pre-treated with a mixture of 5 μmol of hypericin, luminescence substrate with Ruby enhancer and/or two different doses of alkaline phosphatase (AP) for 2 hours at 37° C. and then used to infect CEM-TART cells. HIV p24 was monitored over time. Data is the average of triplicate samples. Closed circles, virus+Ruby+0.0075 U AP+dark; open circles, virus+Ruby+0.18 U AP+dark; closed triangles, virus+hypericin+light; open squares, virus+hypericin+dark; closed squares, untreated virus. Small amounts of alkaline phosphatase are sufficient to catalyze the chemiluminescent substrate in the generation of light for hypericin action;

FIG. 12 is a graph depicting the importance of the presence of an enhancer/substrate mixture for the virucidal effect of alkaline phosphatase-induced hypericin action. Approximately 50 TCID₅₀ of HIVΔtat/Δrev viral stocks were pre-treated with a mixture of 5 μmol of hypericin with or without chemiluminescence substrate with Ruby enhancer and alkaline phosphatase (AP) for 2 hours at 37° C. and then used to infect CEM-TART cells. HIV p24 was monitored over time. Data is the average of duplicate samples. Open circles, virus+hypericin+AP+Enhancer/Substrate+dark; closed circles, virus+hypericin+AP+dark; closed triangle, virus+hypericin+light; open square, virus+hypericin+dark; closed square, untreated virus. The presence of the chemiluminescent substrate with enhancer is necessary for the virucidal action of alkaline phosphatase-induced hypericin action;

FIG. 13 is a graph showing the inhibition of HIV-1Δtat/Δrev growth in virally infected cultures by the concerted action of hypericin, chemiluminescent substrate and alkaline phosphatase. CEM-TART cells infected with 13-20 TCID₅₀ of HIV-1Δtat/Δrev were incubated with hypericin, alkaline phosphatase and a chemiluminescent substrate. The amount of p24 on day 7 post-infection was determined by ELISA. Data given above is the median of triplicates. Not only can hypericin inactivate extracellular virus, but it can also eliminate virus from infected cells.

### DETAILED DESCRIPTION OF THE PREFERRED EMBODIMENTS

The United States and the World are currently experiencing an infectious disease pandemic caused by a novel coronavirus, SARS-CoV-2 that causes COVID-19 (2019-nCoV).

Several compounds with broad anti-viral activity have been described in the literature, but most have been unsuitable for commercial development because of toxicity to the patent. However, there is one class of compounds that do not produce substantial toxicity while at the same time contains strong anti-viral activity. All of these compounds require light to exert their virucidal activity. Such compounds include the light-sensitive plant pigment hypericin and many chemically related compounds from St. John's Wort (Hypericum perforatum) which are able to efficiently kill various enveloped viruses (including but not limited to HIV) in the presence of light. The mechanism of this process is not fully elucidated, but probably includes the induction of singlet oxygen and cross-linking of envelope and capsid proteins of the virus. This wide-ranging antiviral activity was observed in vitro using hypericin concentrations that are not toxic to host cells.

The present inventor has demonstrated that the efficiency of hypericin-induced light-mediated viral inactivation is high for short exposure times, such as during routine tissue culture infection procedures. As observed, these exposure times were sufficient for nearly complete inactivation of the exposed virus, notably HIV and other retroviruses.

However, the virucidal effects of hypericin are minimal if the virus is treated in complete darkness, as in the body. In order to generate an in situ light source, the inventor has developed and patented a “molecular flashlight” [U.S. Pat. No. 7,027,525 to Castor et al], where chemiluminescent substrates combined with enzymes, such as alkaline phosphatase, and emission enhancers or anti-quenchers emit sufficient light to “turn on” the anti-viral properties of the hypericin. It has been demonstrated that hypericin in the presence of this “molecular flashlight” can significantly inactivate H1V in the dark (10⁶ TCID₅₀) in cell culture media in vitro without harming these cells.

Multiple coronavirus strains can be inactivated by a combination of hypericin, chemiluminescent substrates, alkaline phosphatase enzymes and emission enhancers (or anti-quenchers), and optimize the components based on in vitro efficacy and cytotoxicity studies against coronaviruses including those that cause COVID-19.

Formulations for the delivery of this broad-spectrum antiviral coronavirus therapeutic include dry powder pill, gel capsules and nanoparticles (both phospholipid nanosomes for intravenous delivery and polymer nanospheres for oral delivery as well as intranasal delivery), and are evaluated by the in vitro performance (release characteristics, stability, efficacy and toxicity) to inform in vivo studies and further clinical development.

In vivo experiments in animal models of coronavirus are used to establish pharmacokinetics, toxicity, and efficacy, to demonstrate that manufacturing can be scaled in accordance with cGMP guidelines, and establish a regulatory pathway with the FDA to conduct clinical trials on the developed, broad-based, anti-coronavirus through clinical trials for commercialization and stockpiling.

The current COVID-19 pandemic is having a significant impact on the morbidity and mortality of infected patients and is threat to the health and welfare of United States citizens and citizens of other countries around the world. The COVID-19 pandemic is also having a significant impact on the economies and social fabric of all societies around the world.

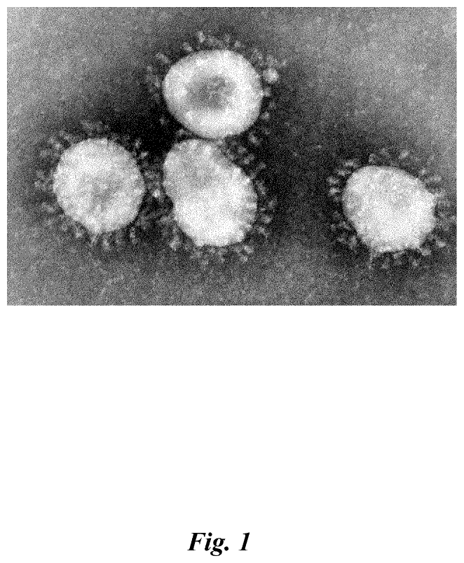

Coronaviruses constitute the subfamily Orthocoronavirinae, in the family Coronaviridae, order Nidovirales, and realm Riboviria. They are lipid-enveloped viruses with a positive-sense single-stranded RNA genome and a nucleocapsid of helical symmetry. The genome size of coronaviruses ranges from approximately 27 to 34 kilobases, the largest among known RNA viruses. The diameter of the virus particles is around 120 nm. The envelope of the virus in electron micrographs appears as a distinct pair of electron dense shells (FIG. 1) with well-defined spikes. Infection begins when the virus enters the host organism and the spike protein attaches to its complementary host cell receptor. After attachment, a protease of the host cell cleaves and activates the receptor-attached spike protein. Depending on the host cell protease available, cleavage and activation allows cell entry through endocytosis or direct fusion of the viral envelope with the host membrane.

In humans, several coronaviruses are known to cause respiratory disease such as Middle East Respiratory Syndrome (MERS), Severe Acute Respiratory Syndrome (SARS) and the most recently discovered SARS-CoV-2 and its variants. These viruses are all genetically related but both SARS (10% fatality rate) and MERS (37.4% fatality rate) being more deadly than COVID-19 (fatality rate ranging of 1.6% to 2.0% but still being established) but much less infectious.

Should the next coronavirus be as infectious as SARS-CoV-2 but have a fatality rate approaching SARS or worse yet MERS, this future pandemic would be much more devastating that the current COVID-19 pandemic if we do not have the tools and capabilities in place to contain and manage coronaviruses. One such tool is vaccines that have been rapidly developed against SARS-CoV-2.

Another tool for our armamentarium is a broad-spectrum therapeutic for multiple coronavirus strains that can be deployed against COVID-19 and can be stockpiled for future outbreaks of coronaviruses.

The development of new antiviral agents is an important and difficult task. In general, compared to other classes of anti-infectives, there are fewer drugs available in the field of antiviral therapy. This is due primarily to features characteristic of viral replication, which occur inside the infected cell and utilizes the cells' molecular machinery. Therefore, there are very few strategies of therapeutic intervention available that do not affect normal cells while simultaneously hindering viral infection. In addition, viral strains resistant to therapy are rapidly emerging, making the available arsenal of drugs significantly less efficient. Some widespread viral diseases (caused by viruses such as coronavirus, Zika, hepatitis C, hemorrhagic fever induced by flaviviruses Ebola, Lassa) are virtually untreatable. Moreover, a number of new and emerging virus's refractory to conventional treatment (such as HIV, West Nile encephalitis, hantavirus) have entered the population in recent decades and this trend is likely to continue.

Therefore, antiviral agents against one or several viruses would be of immense importance to the medical community. Currently, there are no drugs effective against multiple viral agents. There is an immediate need for a class of therapeutics that can effectively eliminate virulent viruses while at the same time not exerting a toxic profile as to render them unusable.

Chemiluminescence-directed antiviral activities of natural and synthesized light-sensitive compounds can be effective in combating a broad range of viral infections. The phenomenon of hypericin-induced viral inactivation has been described in the literature for several decades. Briefly, it has been established that even low concentrations of hypericin and some hypericin-related compounds inactivate most enveloped viruses, including HIV in the absence of significant in vitro cytotoxicity. Apart from inherent phototoxicity, which is neutralized when hypericin is light activated, the benign toxicity profile should be expected for hypericin since it is a major component in St. John's Wort extracts. Unfortunately, the therapeutic use of hypericin for antiviral treatment has been precluded by the major requirement for its action, exposure to visible light.

The efficiency of hypericin-induced light-mediated viral inactivation is so high that even relatively short exposure times, which have occurred during routine tissue culture infection procedures, are sufficient for nearly complete inactivation of the exposed virus, notably HIV and other retroviruses. Upon the realization of this light exposure requirement, it has been shown that fluorescent light provides an even higher degree of hypericin anti-viral activity than visible light, rendering non-infective over 10⁶ TCID₅₀ of HIV. On the contrary, if the virus is treated with hypericin in complete darkness, then the viricidal effects are minimal, if at all detectable. Obviously, one should not expect any benefits from hypericin administration to patients afflicted by viral diseases since there is no light inside the organism. Despite this reasonable assumption, pilot studies of hypericin's benefits for HIV and hepatitis C-infected individuals have been performed with the predictable negative result. The main reason for conducting these trials was hypericin's extremely high anti-viral activity in vitro and its advantageous safety profile. At the same time, hypericin was tested for light-induced inactivation of viruses in blood-related products and this technology has attained a high degree of efficiency.

In order to generate an in situ light source, the inventor has developed and patented a “molecular flashlight” that turns on when novel chemiluminescent substrates are combined with enzymes such as alkaline phosphatase and emission enhancers or anti-quenchers [Castor et al., US Patent, 2006].

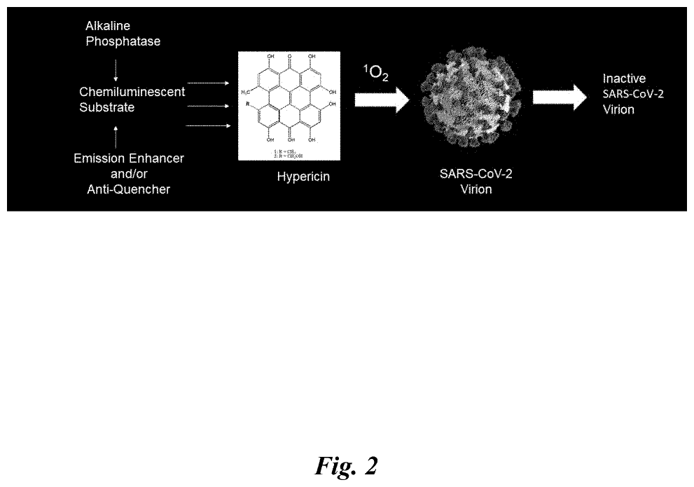

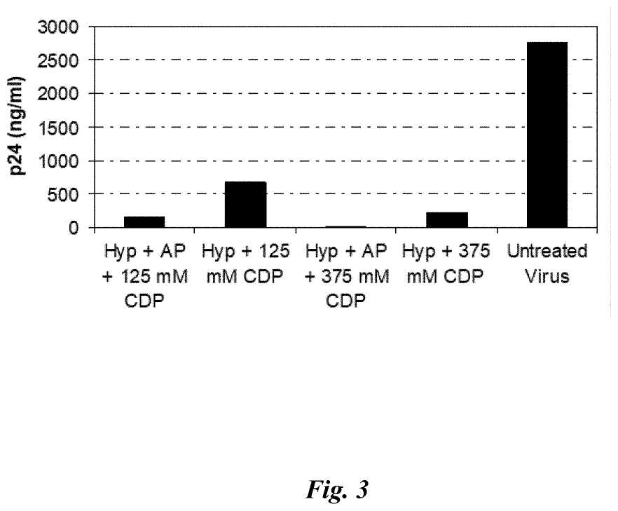

The inventor has demonstrated that the combined use of hypericin, a light-emitting substrate, and an emission enhancer and light-generating enzyme to achieve significant inactivation of enveloped viruses such as HIV-1. FIG. 2 shows a schematic of chemiluminescence-directed inactivation of SARS-CoV-2. FIG. 3 graphically shows the inhibition of HIV-1_{IIIB} growth in infected cultures by a concerted action of hypericin, a chemiluminescent substrate and alkaline phosphatase.

The present invention uses an enzyme normally present inside an organism, namely, alkaline phosphatase and highly active light-emitting substrates, which provide for long and sustained light emission and which provide for (1) a specific wavelength peak of emission and (2) possess an anti-quencher effect thus dramatically lengthening chemiluminescence. Embodiments of the present invention includes the encapsulation of the highly stable compounds as an antiviral pharmaceutical or separate encapsulation of the enzyme or an enzyme-encoding plasmid for more efficient delivery and even the use of resident cellular enzymes in order to induce low-level luminescence (in conjunction with a hypericin-substrate-enhancer complex), which is toxic to viruses but not endogenous cells.

Multiple coronavirus strains can be inactivated by a combination of hypericin, chemiluminescent substrates, alkaline phosphatase enzymes and emission enhancers, and optimize the components based on in vitro efficacy and cytotoxicity studies against coronaviruses including SARS-CoV-2. The chemical components of the antiviral consist of hypericin, chemiluminescent substrates, alkaline phosphatase enzymes and emission enhancers (or anti-quenchers).

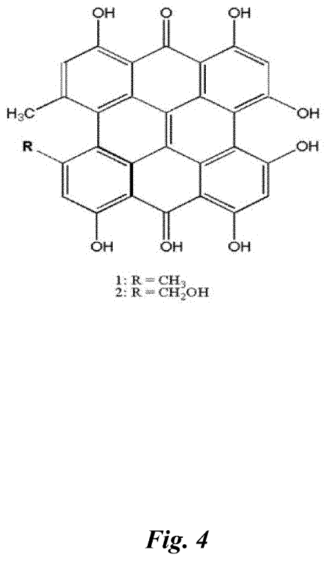

Hypericin [C₃₀H₁₆O₈; Molecular Weight: 504.45; CAS Number: 548-04-9; Aphios Catalog No: APH-20013] is a naphthodianthrone, a red-colored anthraquinone-derivative. Together with hyperforin, it is one of the principal active constituents of St. John's Wort (Hypericum perforatum). Hypericin is a photosensitive pigment found in St. John's Wort. Hypericin is extracted from the flowers and buds of St. John's Wort (Hypericum perforatum) utilizing patented SuperFluids™ CXP technology. Normal phase chromatography is used to separate oils and other components from hypericin. Hypericin is very hydrophobic and soluble in DMSO. It is stable under recommended storage conditions of −20° C. in the absence of light and oxygen. The chemical structure of hypericin is shown in FIG. 4.

In laboratory studies, hypericin has demonstrated anti-retroviral activity using a method of action that does not rely on inhibition, inactivation, or modification of reverse transcriptase. The findings of the one study in particular suggest that, unlike nucleoside analogues, hypericin and pseudohypericin directly inactivate the virions or interfere with assembly or shedding of assembled viral particles. Hypericin is not limited to just coronaviruses and HIV. Studies suggest that it is equally effective at inactivating enveloped viruses such as herpes simplex and influenza A.

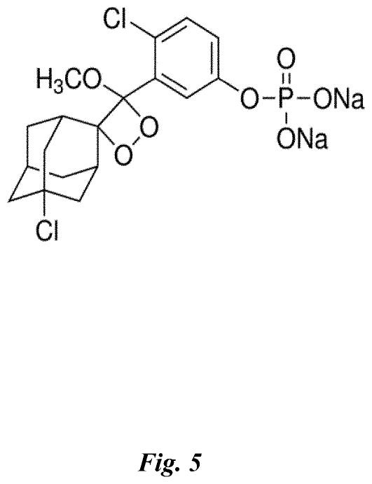

CDP Star®: [C₁₈H₁₉Cl₂O₇Na₂P; Disodium 2-chloro-5-(4-methoxyspiro {1,2-dioxetane-3,2′(5′-chloro)tricyclo[3.3.1.13,7]decan}-4-yl)-1-phenyl phosphate; MW=495.2; CAS No. 160081-629; Sigma-Aldrich Catalog No. C0712] is a sensitive, chemiluminescent substrate of alkaline phosphatase. CDP Star® is suppled as a 0.025M ready-to-use aqueous solution and is stable at 2-8° C. for at least one year. CDP Star® is a registered trademark of Tropix, Inc., Bedford, Mass. and covered under U.S. Pat. No. 5,326,882. FIG. 5 shows the chemical structure of CDP Star®.

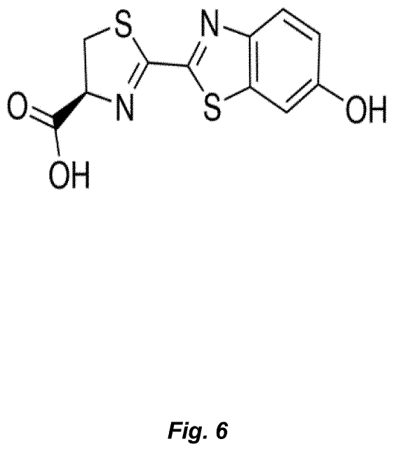

D-Luciferin: [C₁₁H₈N₂O₃S₂; Firefly Luciferin 4,5-Dihydro-2-(6-hydroxy-2-benzothiazolyl)-4-thiazolecarboxylic acid; MW=280.3; CAS No.: 2591-17-5; Sigma-Aldrich Catalog No. L9504] is a specific chemiluminescent substrate for Firefly Luciferase. The excitation is pH dependent, with a maximum of 327 nm at pH 4 and 385 nm at pH 11. The emission profile is identical at both pH's, with a maximum at 537 nm (Bowie, 1978). The dependence of the bioluminescence of the luciferase-luciferin system on Zn²⁺ concentration has been published (DeLuca, 1978). D-luciferin is soluble in methanol (10 mg/mL) and in DMSO (50 mg/mL); storage temperature is −20° C. FIG. 6 shows the chemical structure of D-Luciferin.

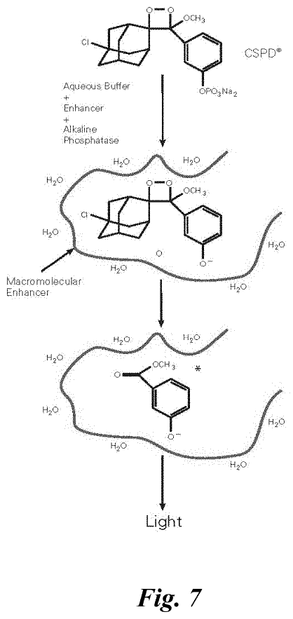

Emission Enhancers or Anti-Quenchers: Aqueous environments reduce chemiluminescent signal intensity of 1,2-dioxetane substrates such as CDP Star® by water-induced quenching; chemiluminescence enhancers increase the emission efficiency of light production by partitioning the water from the site of chemiluminescent signal production. Luminescence enhancers increases the emission efficiency of light production by partitioning the water away from the site of signal production, as shown in FIG. 7.

Tropix enhancers such as Sapphire™, Emerald™, Ruby™, Sapphire-II™, and Emerald-II™ enhancers are essential components of solution-based assays. Enhancers provide signal enhancement with minimal delay of light-emission kinetics. Sapphire™, Emerald™, Ruby™, Sapphire-II™, or Emerald-II™ Enhancers Tropix enhancers shift the wavelength of light emission. Sapphire and Sapphire-II enhancers slightly shift the light emission maximum from 475 nm observed for the dioxetane alone to 461 nm. Emerald and Emerald-II enhancers shift the light emission maximum to 542 nm, while Ruby™ enhancer shifts the light emission maximum to 620 nm. Although the signal intensity obtained with Emerald and Emerald-II enhancers is much greater compared to other enhancers, use of Sapphire and Sapphire-II enhancers produces a wider dynamic range since photodetector saturation is less likely to occur. The Emerald or Emerald-II enhancer is the optimum choice for applications requiring maximum signal intensity.

DOE (Design of Experiments) are utilized to evaluate the feasibility of different permutations and combinations of this 4-component system to inactivate several different coronaviruses and measure their cytotoxicity against normal cells. Based on these measurements, a cohort of broad-spectrum therapeutics for multiple coronavirus strains are selected based on a Selective Index (SI)=50% Cytotoxicity/50% Efficacy=CC₅₀/EC₅₀ for further development.

Experiments are conducted to determine efficacy against four (4) coronaviruses—two low pathogenicity human coronaviruses, one well-studied mouse coronavirus and the novel coronavirus, SARS-CoV-2 under BSL-3 laboratory conditions.

Mildly pathogenic human coronavirus (HCV) strain 229E (ATCC VR-740) and Betacoronavirus 1, strain OC43 (ATCC® VR-1558™) are obtained from the ATCC. HCV 229E is able to grow in human cell lines such as MRC-5 and produces CPE consisting of rounding and sloughing of cells. MRC5 (ATCC CCL-171) is a human lung fibroblastic cell line obtained from a normal 14-week-old male fetus. It supports the replication of a number of respiratory viruses including human coronaviruses. MRC-5 cells, 80-90% confluent, are infected at a relatively high multiple-of-infection (MOI of 0.1 to 0.2) and the virus is harvested 24-48 hours post infection before CPE is visible. HCV OC43 shows no cross reactivity with HCV strain 229E, and is able to grow in human cell lines such as HCT-8 (ATCC CCL-244) and produces CPE consisting of vacuolation and sloughing of cells. Mouse hepatitis virus strain MHV-A59, a mouse coronavirus, is also obtained from ATCC and grown in NCTC clone 1469, a mouse liver cell line, in which it produces CPE consisting of syncytia, rounding and sloughing of cells. Human coronavirus SARS-CoV-2, the causative agent of COVID-19, is obtained from the NIH or CDC. The virus is grown in human cell lines such as Vero (ATCC CCL-81) which produces CPE consisting of rounding and detachment of cells.

The virus stocks generated above are titrated in 96 well plates using standard TCID₅₀ procedure on their respective host cells. Briefly, confluent monolayers of the host cells are infected with serial log dilutions of the virus in replicates of 8. CPE is monitored for 5-10 days and the number of wells showing CPE are used to calculate the TCID₅₀ by the Karber method. The duration of the assay that gives the highest titers are optimized initially. Additionally, virus titrations are performed by qPCR of viral nucleic acids and ELISA and/or lateral flow assays for viral antigens in the culture supernatants in the TCID₅₀ assay.

Cytotoxicity and interference studies are performed in parallel with the efficacy studies by the various combinations of the four-component systems to ensure that any efficacy observed is not due to the effects of these compounds on the host cells. Cytotoxicity studies are performed by treating the cells with different doses of the combinations of drugs for the same durations as the efficacy assays. At the end of the treatment periods, the cells are visually examined for morphological changes and the metabolic activity assayed by a metabolic assay such as CellTiter 96® AQueous One by Promega. The non-toxic doses for each of the combinations are determined by this method.

Interference studies are performed to determine if the treatment of the host cells by these compounds compromises their ability to support viral replication. The optimal way to perform these studies is to neutralize these compounds in the same way as is done for the efficacy studies. If that is not feasible, the cells are treated with the compounds alone (in the absence of the virus) for the same duration as the efficacy study and then the compounds are washes off, followed by titration of a stock of the untreated virus. The titer of the virus stock obtained after pretreatment of the cells are compared with the untreated controls and any differences in titers are evaluated. Assay conditions are established where the differences are not greater than the normal range of the assay variation.

Formulations for the delivery of this broad-spectrum antiviral coronavirus therapeutic include dry powder pill, gel capsules and nanoparticles (both phospholipid nanosomes for intravenous delivery and polymer nanospheres for oral delivery, and intranasal delivery), and are evaluated by the in vitro performance (release characteristics, stability, efficacy and toxicity) to inform in vivo studies and further clinical development.

The four components of the proposed antiviral therapeutic have different physical and chemical characteristics. They must also be either delivered separately or kept in nonreactive components until delivered to their viral targets, e.g. coronaviruses. Formulation development and drug delivery are thus of critical importance to the photoluminescence therapeutic.

For critical care patients in an ICU setting, the therapy will be delivered by intravenous (IV) administration. For non-hospitalized cases with milder symptoms or asymptomatic patients, the therapeutic will be administered orally (po) or intranasally (in).

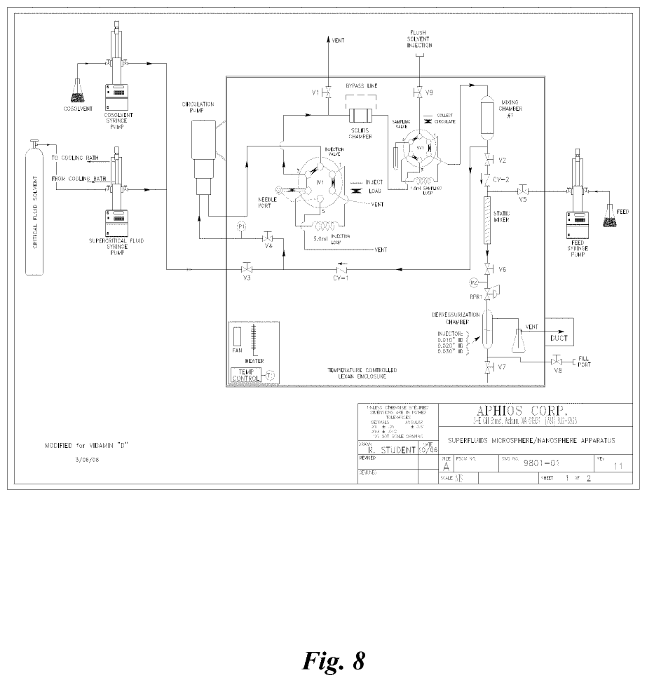

For intravenous (IV) administration, the combination therapeutic is preferably encapsulated in long-circulating, pegylated, small uniform phospholipid liposomes (nanosomes). In these nanosomes, hypericin and other hydrophobic components are encapsulated in the lipid bilayer, and chemiluminescent substrates and enzymes are encapsulated in the aqueous core of these nanoparticles. Thus, nanosomes are used to isolate reacting components until they are delivered in the blood stream. The nanosomes are also utilized for the enhanced cellular delivery into mammalian cells hosting the virus such as HIV-1 or SARS-CoV-2. The apparatus for creating phospholipid nanosomes is schematically illustrated in FIG. 8.

In an embodiment of this invention, one of three SFS: CO₂, near-critical propane, or an alternative fluorocarbon solvent is used. Based on prior experience, we use SFS propane and 20% ethanol at 3,000 psig and 40° C. Synthetic lipids, DPPC (1,2-dipalmitoyl-sn-glycero-3-phosphocholine) and DSPE-PEG-2000 (1,2-dipalmitoyl-sn-glycero-3-phospho-ethanolamine-N-[methoxy(polyethylene glycol)-2000] (ammonium salt)) from Lipoid GmbH of Germany are selected for the nanoencapsulation experiments. Synthetic lipids are selected since they were uniform in-chain length and saturation of bonds; however, natural soy and egg lipids can be utilized. The molar ratio of total lipid to drug ranges from ˜5:1 to ˜20:1. Hydrophobic components are encapsulated in phospholipid nanosomes in the modified SFS-CFN apparatus shown in FIG. 8. The process produces three different nanosomes by various processing steps in the range of 100 to 200 (±50) nm. The process is inherently a sterile process so nanosomal suspensions of this size range are the pre-final drug product. If necessary, nanosomes are filtered by a 0.22 μm filter as a final sterilization process. Nanosomes are then lyophilized to produce the final drug product. Chemical content and loading efficiencies are determined by HPLC.

In another embodiment of this invention for oral (p.o.) administration, hypericin and hydrophobic as well as hydrophilic components are nanoencapsulated in hydrophobic polymeric nanospheres, and lyophilized into a dry powder. For manufacturing polymer nanospheres, we utilize proprietary SuperFluids™ Polymer Nanospheres (SFS-PNS) technology. For hydrophobic biodegradable polymers, we utilize pharmaceutical-grade PLGA [poly (D,L-lactide-co-glycolide) 50:50] polymer (Resomer® RG-502, Boehringer Ingelheim KG) and/or PCL [Polycaprolactone] purchased from Sigma Aldrich. In the SFS-PNS nanoparticles process, the biodegradable polymer and hydrophobic components are placed in the solids chamber, the high-pressure circulation (HPC) loop is pressurized with the SuperFluids aka SFS (supercritical, critical or near-critical fluids with or without small quantities of polar cosolvents), e.g., a mixture of propane:ethanol::80:20 at 3,000 psig and 40° C.) and circulated for a specific time, e.g., 30 minutes. The hydrophobic-enriched SFS stream is then mixed with a feed stream containing hydrophilic constituents that are continuously decompressed through a nozzle into an aqueous buffer (e.g., 10% sucrose). In another modification of the SFS-CFN process shown in FIG. 8, hydrophobic polymers and hydrophobic components are dissolved in an SFS at a specific pressure and temperature and mixed with an ethanolic or aqueous solution at the same pressure and the same or different temperature.

The buffer may contain a cross-linking again such as polyvinyl alcohol (PVA) to stiffen and make firmer the nanoparticles. The buffer can also be used to contain the hydrophilic constituents of the therapeutic. The pH and ionic strength will be selected for the optimal formation of nanoparticles in terms of size, uniformity and chemical content. As a result of decompression, hydrophobic molecules come out of solution, thermodynamically self-assemble and polymer nanospheres are formed encapsulating hypericin and both hydrophobic and hydrophilic components of the antiviral therapeutic. The nanospheres formation process is inherently sterile. If necessary, nanospheres are filtered by a 0.22 μm filter as a final sterilization process. Nanospheres are then lyophilized to produce the final drug product. Chemical content and loading efficiencies are determined by HPLC.

In another embodiment of this invention for oral (p.o.) administration, hypericin and hydrophobic components are nanoencapsulated in hydrophobic polymeric nanospheres, and mixtures of these nanoparticles and aqueous components are lyophilized into a dry powder.

The lyophilized powder is used to dry fill size 0, 2 and 3 capsules, and making tablets.

Both the nanosomes and nanospheres can be used for the intranasal delivery of the antiviral therapeutic.

The nanoparticles (nanosomes and nanospheres) are tested for in vitro efficacy and toxicity and characterized by size distribution, mean size, and standard deviation are analyzed by a submicron particle analyzer (Coulter Electronics Model N4MD) with a range of 30 Angstroms to 3 microns. Drug-content and encapsulation efficiency are determined by HPLC using a Phenomenex Luna 5-micron C18(2) 15-cm×4.6 mm HPLC column at a temperature of 30° C., flow rate of 1.0 mL/min and an injection volume of 20 μL with monitoring at a wavelength of 265 nm. In vitro release kinetics of nanoparticles are carried out by placing a sample of nanoparticles in PBS buffer (pH=7.4) or plasma at 25° C. and 37° C. Stability studies are conducted on nano-encapsulated and naked drug samples at 4° C., 25° C. and 40° C. following ICH guidelines in terms of (i) physical appearance; and (ii) drug content. Statistical analysis of the data sets is performed using SYSTAT®.

### EXAMPLES

### Example 1: Inactivation of HIV-1 Replication by Concerted Action of Hypericin and Visible Light

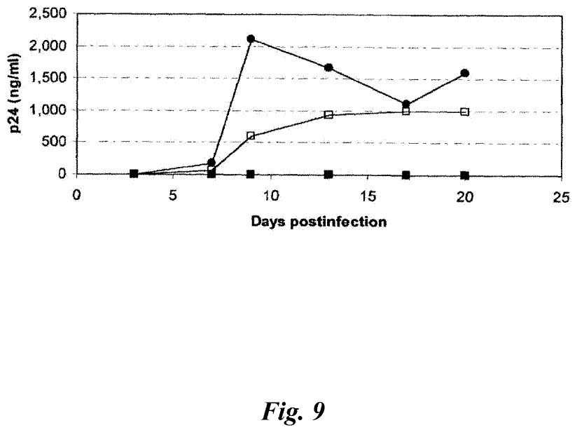

HIV-1 Δtat/Δrev stocks were pre-treated with hypericin (5 μmol) and then exposed to visible light (hypericin+light) for 1 hour at 37° C. or kept in the dark for the same amount of time (hypericin+dark). These stocks were then used for infection of CEM-TART cells (CEM cells expressing HIV-1 tat and rev). The data is listed in Table 1 and shown in FIG. 9. Viral stocks exposed to light without hypericin (data not shown) or treated with hypericin in the dark, grew similarly. Greater than 99% of the virus was inactivated when exposed to hypericin and light, even after 20 days of culture.

TABLE 1
Inactivation of HIV-1 Replication by Concerted
Action of Hypericin and Visible Light

| Amount of | HIV p24 concentration (ng/ml) | | | | | | |
|---|---|---|---|---|---|---|---|
| HIV used, | Days Post-infection | | | | | | |
| treated by | 0 | 3 | 7 | 9 | 13 | 17 | 20 |
| 50 TCID₅₀, | 1 | 0.87 | 1.57 | 8.12 | 2.04 | 2.20 | 6.85 |
| 5 μm hyper- | | | | | | | |
| icin + light | | | | | | | |
| 50 TCID₅₀, | 1 | 1.84 | 60.53 | 606.57 | 935.42 | 1000 | 1000.61 |
| 5□m hyper- | | | | | | | |
| icin + dark | | | | | | | |
| 50 TCID₅₀ | 1 | 5.96 | 180.35 | 2113.7 | 1659.86 | 1116.11 | 1599.33 |
| virus only | | | | | | | |

### Example 2: Virucidal Effect of Hypericin and Chemiluminescence-Inducing Compounds

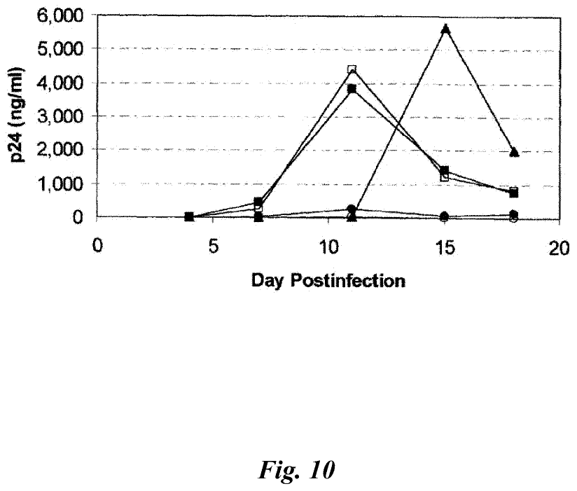

Approximately 50 TCID₅₀ (1 ng of p24) of HIV-1Δtat/Δrev viral stocks treated with various combinations of hypericin (5 μmol, Hyp), chemiluminescence substrate with enhancer (2.5 mmol) and alkaline phosphatase (0.18 U, AP) for 1 hour at 37° C. were used to infect CEM-TART cells, which were then passaged in a conventional manner (samples taken every 3-4 days before replacing tissue culture media). The contents of each experimental sample are shown in the left column of Table 2. Each mixture was tested in duplicate. HIV replication was measured by the amount of p24 capsid protein in the culture media. Hypericin exposed to light (hyp+light) is a positive control of viral inhibition. Emerald™ enhancer was used. The averaged data of duplicate samples is shown graphically in FIG. 10. Incubating virus with hypericin, alkaline phosphatase and a chemiluminescent substrate with enhancer results in equivalent virus inactivation as when hypericin and visible light are used (99.3% virus reduction with chemiluminescent substrate and enhancer versus 99.4% virus reduction with visible light on day 7 post-infection).

TABLE 2
Virucidal Effect of Hypericin and Chemiluminescence-
Inducing Compounds

| | HIV p24 concentration (ng/ml) | | | | | |
|---|---|---|---|---|---|---|
| Mixture | Days Post-infection | | | | | |
| components | 0 | 4 | 7 | 11 | 15 | 18 |
| Hyp + Enhancer + | 1 | 1.4 | 2.3 | 37 | 12 | 2.8 |
| AP + dark | 1 | 1.7 | 5.3 | 48 | 15 | 13 |
| Hyp + Enhancer + | 1 | 2.5 | 39.5 | 327 | 104 | 11 |
| dark | 1 | 3.1 | 42 | 195 | 45 | 178 |
| Hyp + AP + | 1 | 3.8 | 251 | 5774 | 1506 | 1307 |
| dark | 1 | 2.7 | 269 | 3088 | 917 | 329 |
| Untreated virus | 1 | 3.3 | 462 | 3838 | 1405 | 766 |
| Hyp + light | 1 | 1 | 4.1 | 44.7 | 11343 | 4011 |
| | 1 | 1 | 1.2 | 2.2 | 1.9 | 1 |

### Example 3: The Level of Virucidal Effect of Chemiluminescence-Induced Hypericin Action is Dependent on the Concentration of the Luminogenic Enzyme

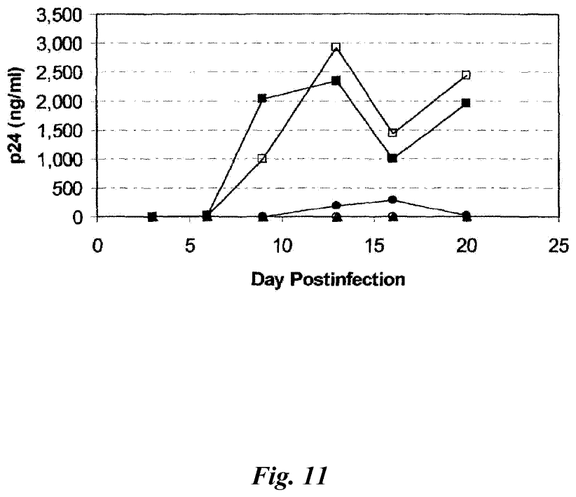

Approximately 50 TCID₅₀ (1 ng of p24) of HIV-1Δtat/Δrev viral stocks were pre-treated with a mixture of hypericin (5 μmol, Hyp), luminescence substrate with Ruby enhancer (Ruby) and different doses of alkaline phosphatase (AP) for 2 hours at 37° C. and then used to infect CEM-TART cells. Cells culture media was exchanged every 3-4 days and samples for p24 analysis were taken at the same time. HIV replication was measured by the amount of p24 capsid protein in the culture media. Samples treated with hypericin and exposed to the light (hyp+light) was a positive control of viral inhibition; unexposed samples (hyp+dark) were used as negative control. Samples were tested in triplicate. The raw data are listed in Table 3 and the averaged data is shown in FIG. 11. Small amounts of alkaline phosphatase are sufficient to catalyze the chemiluminescent substrate in the generation of light for hypericin action.

TABLE 3
The level of Virucidal Effect of Chemiluminescence-Induced Hypericin
Action is Dependent on the Concentration of the Luminogenic Enzyme

| | HIV p24 concentration (ng/ml) | | | | | | |
|---|---|---|---|---|---|---|---|
| Mixture | Days Post-infection | | | | | | |
| components | 0 | 3 | 6 | 9 | 13 | 16 | 20 |
| Hyp + Ruby + 0.0075 U | 1 | 0.05 | 0.2 | 7.2 | 1.9 | 1.4 | 5.1 |
| AP + dark | 1 | 0.69 | 1 | 3.5 | 1.9 | 1.3 | 4.3 |
| | 1 | 0.05 | 2 | 10 | 601 | 852 | 29 |
| Hyp + Ruby + 0.18 U | 1 | 0.05 | 0.05 | 3.1 | 0.05 | 0.05 | 3.6 |
| AP + dark | 1 | 0.05 | 0.05 | 3 | 1.85 | 0.05 | 2.6 |
| | 1 | 0.05 | 0.05 | 3.3 | 3.6 | 1.4 | 2.9 |
| Hyp + dark | 1 | 0.05 | 12.13 | 831 | 3,501 | 1543 | 2,789 |
| | 1 | 0.05 | 11 | 1,120 | 3,270 | 923 | 2,775 |
| | 1 | 0.05 | 19.34 | 1,055 | 1,985 | 1,870 | 1,763 |
| Hyp + light | 1 | 0.05 | 0.05 | 3 | 2.9 | 0.05 | 6.6 |
| Untreated virus | 1 | 0.124 | 19.25 | 2,032 | 2,352 | 1,008 | 1,958 |

### Example 4: Presence of Enhancer/Substrate Mixture is Essential for Virucidal Effect of Alkaline Phosphatase-Induced Hypericin Action

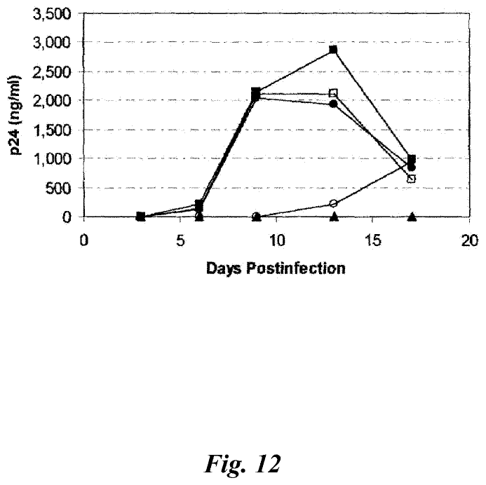

Approximately 50 TCID₅₀ of HIV-1Δtat/Δrev viral stocks were pre-treated with a mixture of hypericin (5 μmol, Hyp), with or without chemiluminescence substrate pre-mixed with a Ruby™ enhancer (1.25 mmol, Ruby) and alkaline phosphatase (0.036 U, AP) for 2 hours at 37° C. and then used to infect CEM-TART cells. HIV replication was measured by the amount of p24 capsid protein in the culture media. Samples treated with hypericin and exposed to the light (Hyp+light) served as a positive control of viral inhibition, unexposed samples (Hyp+dark) were used as negative controls. Samples were tested in duplicate. The raw data are listed in Table 4 and the averaged data is shown in FIG. 12. The presence of the chemiluminescent substrate with enhancer is necessary for the virucidal action of alkaline phosphatase-induced hypericin action.

TABLE 4
Presence of Enhancer/Substrate Mixture is Essential for Virucidal
Effect of Alkaline Phosphatase-Induced Hypericin Action

| | HIV p24 concentration (ng/ml) | | | | | |
|---|---|---|---|---|---|---|
| Mixture | Days Post-infection | | | | | |
| components | 0 | 3 | 6 | 9 | 13 | 17 |
| Hyp + AP + | 1 | 0.05 | 0.05 | 2.8 | 214 | 945.5 |
| Enhancer/ | 1 | 0.05 | 0.05 | 6 | 210 | 958.2 |
| Substrate + dark | | | | | | |
| Hyp + AP + | 1 | 1.4 | 74.4 | 1,908 | 1,925 | 705.4 |
| dark | 1 | 5.4 | 196 | 2,167 | 1,948 | 958.2 |
| Hyp + dark | 1 | 3.3 | 149 | 2,191 | 2,322 | 662.5 |
| | 1 | 4 | 144 | 2,031 | 1,909 | 609.5 |
| Hyp + light | 1 | 0.05 | 0.05 | 0.05 | 0.05 | 0.05 |
| Untreated virus | 1 | 2.3 | 222 | 2,150 | 2,868 | 976 |

### Example 5: Inhibition of HIV-1ΔTat/ΔRev Growth in Infected Cultures by a Concerted Action of Hypericin, Chemiluminescent Substrate and Alkaline Phosphatase

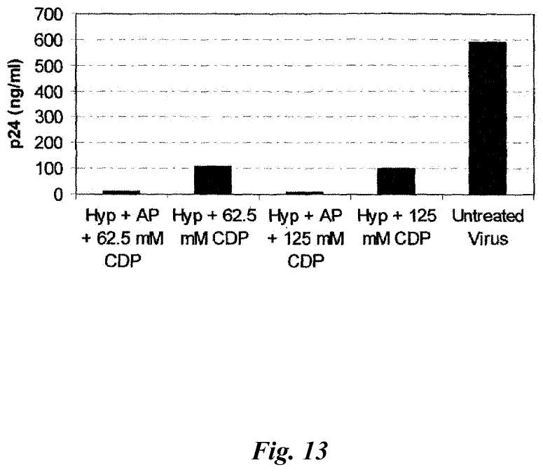

CEM-TART cells infected with 13-20 TCID₅₀ of HIV-1Δtat/Δrev were incubated with hypericin (5 μmol), alkaline phosphatase (Calbiochem, 0.18 U) and chemiluminescent substrate CDP-Star in the concentrations shown. Tissue culture media was replaced every 3-4 days. HIV replication was measured by the amount of p24 capsid protein in the culture media from day 7. The median data of three replicates are listed in Table 5 and shown in FIG. 13. Not only can hypericin inactivate extracellular virus, but it can also eliminate virus from infected cells.

TABLE 5
Inhibition of HIV-1Δtat/Δrev Growth in Infected
Cultures by a Concerted Action of Hypericin, Chemiluminescent
Substrate and Alkaline Phosphatase

| HIV-1 Δtat/Δrev-infected CEM-TART | p24 concentration (ng/ml) on |
|---|---|
| cells treated with: | day 7 post-infection |
| Hypericin, alkaline phosphatase, 62.5 μm | 11 |
| CDP | |
| Hypericin, 62.5 μm CDP | 110 |
| Hypericin, alkaline phosphatase, 125 μm | 8.8 |
| CDP | |
| Hypericin, 125 μm CDP | 100 |
| Untreated virus | 592 |

### Example 6: Inhibition of HIV-1_{IIIB} Growth in Infected Cultures by a Concerted Action of Hypericin, Chemiluminescent Substrate and Alkaline Phosphatase

CEM-SS cells were infected with 13-20 TCID₅₀ of HIV-1_{IIIB}, and then incubated with hypericin (5 μmol), alkaline phosphatase (Calbiochem, 0.18 U) and chemiluminescent substrate CDP in the concentrations shown. Tissue culture media was replaced every 3-4 days. HIV replication was measured by the amount of p24 capsid protein in the culture media from day 7. The median data of three replicates are listed in Table 6 and shown in FIG. 2. The alkaline phosphatase-induced hypericin action is also effective against fully competent virus.

TABLE 6
Inhibition of HIV-1_{IIIB} Growth in Infected Cultures
by a Concerted Action of Hypericin, Chemiluminescent
Substrate and Alkaline Phosphatase

| HIV-1_{IIIB}-infected CEM-SS | p24 concentration (ng/ml) on |
|---|---|
| cells treated with: | day 7 post-infection |
| Hyp + AP + 125 μm CDP + dark | 161 |
| Hyp + 125 μm CDP + dark | 664 |
| Hyp + AP + 375 μm CDP + dark | 6 |
| Hyp + 375 μm CDP + dark | 219 |
| Untreated virus | 2766.7 |

### Example 7: Virucidal Effect of Chemiluminescence-Induced Hypericin Action in the Presence of Luminescence Enhancer

We evaluated the level of virucidal effect of chemiluminescence-induced hypericin action in the presence of a luminescence enhancer. 50 TCID₅₀ (1 ng of p24) of HIV-1ΔtatΔrev viral stocks were pre-treated with a mixture of hypericin (5 μmol, Hyp), luminescence substrate with Ruby™ enhancer (Ruby) and different doses of alkaline phosphatase (AP) for 2 hours at 37° C. and then used to infect CEM-TART cells. Cells culture media was exchanged every 3-4 days and samples for p24 analysis were taken at the same time. HIV replication was measured by amount of p24 capsid protein in the culture media. Sample treated with hypericin exposed to the light (*) was a positive control of viral inhibition; unexposed samples (Hyp only) were used as negative control. These experiments were conducted in triplicate and showed good reproducibility and inactivation dependent on the concentration of the luminogenic enzyme (Table 7).

TABLE 7
Virucidal Effect of Chemiluminescence-Induced Hypericin Action

| | HIV p24 concentration (ng/ml) as a function of | | | | | | |
|---|---|---|---|---|---|---|---|
| | time after infection (days) | | | | | | |
| Mixture components | 0 | 3 | 6 | 9 | 13 | 16 | 20 |
| intact HIV virus | 1 | 0.124 | 19.25 | 2,032 | 2,352 | 1,008 | 1,958 |
| Hyp only₁ | 1 | 0.05 | 12.13 | 831 | 3,501 | 1543 | 2,789 |
| Hyp only₂ | 1 | 0.05 | 11 | 1,120 | 3,270 | 923 | 2,775 |
| Hyp only₃ | 1 | 0.05 | 19.34 | 1,055 | 1,985 | 1,870 | 1,763 |
| Hyp the light* | 1 | 0.05 | 0.05 | 3 | 2.9 | 0.05 | 6.6 |
| Hyp Ruby (0.0075 μmol AP)₁ | 1 | 0.05 | 0.2 | 7.2 | 1.9 | 1.4 | 5.1 |
| Hyp Ruby (0.0075 μmol AP)₂ | 1 | 0.69 | 1 | 3.5 | 1.9 | 1.3 | 4.3 |
| Hyp Ruby (0.0075 μmol AP)₃ | 1 | 0.05 | 2 | 10 | 601 | 852 | 29 |
| Hyp Ruby (0.18 μmol AP)₁ | 1 | 0.05 | 0.05 | 3.1 | 0.05 | 0.05 | 3.6 |
| Hyp Ruby (0.18 μmol AP)₂ | 1 | 0.05 | 0.05 | 3 | 1.85 | 0.05 | 2.6 |
| Hyp Ruby (0.18 μmol AP)₃ | 1 | 0.05 | 0.05 | 3.3 | 3.6 | 1.4 | 2.9 |

The detailed description set forth above is provided to aid those skilled in the art in practicing the present invention. However, the invention described and claimed herein is not limited in scope by the specific embodiments herein disclosed. The embodiments are intended as illustrations of several aspects of the invention. Any equivalent embodiments are intended to be within the scope of this invention. Various modifications of the invention in addition to those shown and described herein become apparent to those skilled in the art from the foregoing description which do not depart from the spirit or scope of the present inventive discovery. Such modifications are also intended to fall within the scope of the appended claims.

What is claimed is:

1. A dosage form of an antiviral complex, comprising an antiviral light sensitive compound, a phosphatase enzyme, a chemiluminescence substrate, and a chemiluminescence enhancer, wherein said antiviral light sensitive compound is selected from the group consisting of hypericin, pseudohypericin, and hypocrellin; and wherein said chemiluminescence substrate is a water soluble 1,2-dioxetane; wherein said chemiluminescence enhancer is a quaternary ammonium homopolymer with or without a fluorescent organic compound encapsulated in phospholipid nanosomes; and wherein the said antiviral light sensitive compound is encapsulated in the lipid bilayer of the nanosomes and the said phosphatase enzyme, chemiluminescence substrate, and chemiluminescence enhancer are co-encapsulated in the aqueous core of the nanosomes.

2. The dosage form of claim 1, wherein said antiviral light sensitive compound is hypericin.

3. The dosage form of claim 1, wherein said phosphatase enzyme is alkaline phosphatase or an analog thereof.

4. The dosage form of claim 1, wherein said 1,2-dioxetane is disodium 3-(4-methoxyspiro[1,2-dioxetane-3,2′-(5′-chloro)-tricyclo [3.3.1.1_{3,7}] decan]-4-yl) phenyl phosphate (CSPD).

5. The dosage form of claim 1, wherein said 1,2-dioxetane is disodium 2-chloro-5-(4-methoxyspiro [1,2-dioxetane-3,2′-(5′chloro)-tricyclo [3.3.1.1_{3,7}] decan]-4-yl)-1-phenyl phosphate (CDP-STAR).

6. The dosage form of claim 1, wherein said chemiluminescence enhancer is poly (benzyltributyl) ammonium chloride (SAPPHIRE-II).

7. The dosage form of claim 6, wherein said chemiluminescence enhancer is poly (benzyltributyl) ammonium chloride and sodium fluorescein (EMERALD-II).

8. The dosage form of claim 1 wherein the nanosomes are pegylated.

9. The dosage form of claim 1 wherein the nanosomes are sterilized.

10. The dosage form of claim 1 wherein the nanosomes are lyophilized.

12. A dosage form of an antiviral complex, comprising an antiviral light sensitive compound, a phosphatase enzyme, a chemiluminescence substrate, and a chemiluminescence enhancer, wherein said antiviral light sensitive compound is selected from the group consisting of hypericin, pseudohypericin, and hypocrellin; and wherein said chemiluminescence substrate is a water soluble 1,2-dioxetane; wherein said chemiluminescence enhancer is a quaternary ammonium homopolymer with or without a fluorescent organic compound encapsulated in phospholipid nanosomes; and wherein the said antiviral light sensitive compound phosphatase enzyme, chemiluminescence substrate, and chemiluminescence enhancer are co-encapsulated in hydrophobic polymer nanospheres.

13. The dosage form of claim 12 wherein the said biodegradable polymer and hydrophobic antiviral light sensitive compound are placed in the solids chamber, the high-pressure circulation (HPC) loop is pressurized with the SuperFluids aka SFS (supercritical, critical or near-critical fluids with or without small quantities of polar cosolvents), e.g., a mixture of propane:ethanol::80:20 at 3,000 psig and 40° C.) and circulated for a specific time, e.g., 30 minutes and wherein the hydrophobic-enriched SFS stream is then mixed with a feed stream containing hydrophilic constituents said phosphatase enzyme, a chemiluminescence substrate, and a chemiluminescence enhancer that are continuously decompressed through a nozzle into an aqueous buffer (e.g., 10% sucrose).

14. The dosage form of claim 13 wherein hydrophobic polymers and hydrophobic components are dissolved in an SFS at a specific pressure and temperature and mixed with an ethanolic or aqueous solution at the same pressure and the same or different temperature.

15. The dosage form of claim 13 wherein the buffer contains a cross-linking again such as polyvinyl alcohol (PVA) to stiffen and make firmer the nanoparticles.

16. The dosage form of claim 13 wherein the buffer contains the hydrophilic constituents of the therapeutic.

17. The dosage form of claim 13 wherein the pH and ionic strength will be selected for the optimal formation of nanoparticles in terms of size, uniformity and chemical content.

18. The dosage form of claim 13 wherein the polymer nanospheres are lyophilized.

19. The dosage form of claim 12 wherein the nanospheres are orally and intranasally administered for the treatment of COVID-19.

20. A method of treating COVID-19 by the intravenous administration of an antiviral complex, comprising an antiviral light sensitive compound, a phosphatase enzyme, a chemiluminescence substrate, and a chemiluminescence enhancer, wherein said antiviral light sensitive compound is selected from the group consisting of hypericin, pseudohypericin, and hypocrellin; and wherein said chemiluminescence substrate is a water soluble 1,2-dioxetane; wherein said chemiluminescence enhancer is a quaternary ammonium homopolymer with or without a fluorescent organic compound encapsulated in phospholipid nanosomes; and wherein the said antiviral light sensitive compound is encapsulated in the lipid bilayer of the nanosomes and the said phosphatase enzyme, chemiluminescence substrate, and chemiluminescence enhancer are co-encapsulated in the aqueous core of the nanosomes.
